(19) United States
(12) Patent Application Publication
(10) Pub. No.: US 2025/0288551 A1
(43) Pub. Date: Sep. 18, 2025

(54) Malt Supplement Containing Oleocanthal, with Anti-inflammatory, Antioxidant, and Anti-cancer Properties

(71) Applicant: Gorgio International Holding LLC, Tbilisi (GE)

(72) Inventor: Sepehr Aghahosseini, Lüneburg (DE)

(73) Assignee: Gorgio International Holding LLC, Tbilisi (GE)

(21) Appl. No.: 18/860,664

(22) PCT Filed: Jul. 27, 2022
(86) PCT No.: PCT/IB2022/056917

§ 371 Date: Oct. 27, 2024

(51) Int. Cl. A61K 31/222; A61K 9/19; A61K 31/355; A61K 36/8998

(52) U.S. Cl. CPC A61K 31/222; A61K 9/19; A61K 31/355; A61K 36/8998; A61K 2236/333

(57) ABSTRACT

The invention entitled “Malt supplement Containing Oleocanthal, with Anti-inflammatory, Antioxidant and Anti-cancer Properties” is in the field of extracting and formulating a compound used in food, pharmaceutical, and supplement industries with special applications in biomedicine. Oleocanthal is a polyphenol with significant medicinal and nutritional value, however, no method has been proposed yet for its economic extraction while preserving properties. Moreover, this substance is lost in the debittering process and becomes useless. In this invention, an innovative supercritical process with triple solvents is used to extract quality oleocanthal from the olive fruits at a high rate and the temperature and pressure of 59° C. and 2 MPa, respectively. Interestingly, in this process, the materials used can be collected and used again. The addition of oleocanthal in the boiling stage of the malt extraction process leads to the production of a cost-effective and quality malt drink supplement with antioxidant and anti-cancer properties.

### TECHNICAL FIELD

Extracting and formulating a compound used in food, pharmaceutical, and supplement industries with special applications in biomedicine.

### BACKGROUND ART

Olive fruit and leaves contain large amounts of polyphenols, including tyrosol, oleuropein, oleosin, oleocanthal, etc., which have great medicinal value. So far, many dietary and pharmaceutical supplements have been formulated and synthesized from Oleuropein (CN100387126c). Some of the properties of these herbal polyphenols are as follows:
Inhibition of cancer cells through the upregulation of P53 expression (US20090048187A1)
Increase AMPK | (CN100387126c)
Decrease mTOR | (WO2013025498A1, CN103929959A)
Skin protection | (WO2001076579A1, WO2015002872A1, U.S. Pat. No. 9,107,853B2)
Antiviral and antibacterial | (WO20014013122A1, US20140072660A1, U.S. Pat. No. 5,494,661A)

After oleuropein, tyrosol and hydroxytyrosol are the most abundant polyphenols in olives, whose anti-cancer and anti-metastasis properties have already been patented and different supplements and medicines have been produced from them.

Oleosin is another polyphenol in olives, which has been claimed to have antioxidant, antimutagenic, anticarcinogenic, and anti-inflammatory properties. It has been used in the synthesis of supplements in patents (WO2002022145A2), (WO2013148324A1), and (WO2017122034A1).

In supplement synthesis patents (WO2002022145A2, WO2013148324A1, and WO2017122034A1), the antioxidant, antimutagenic, anticarcinogenic, and anti-inflammatory properties of oleosin as another polyphenol in olives have been claimed.

Oleocanthal is a polyphenol that is extracted from virgin olive oil in small amounts of 0.2 to 0.002%. The extraction of this polyphenol through supercritical methods with methanol:water (80:20, v/v) and water:alcohol:acetone solvents has been registered in patents WO2006122128A2 and US20090076142A1, respectively. In 2015, in China, under the patent number (CN103483196B), the multi-step extraction methods of oleocanthal have been claimed.

In fact, in this method, the oil is induced to be aldehydic (sweet oil), and then it is extracted from virgin oil by propane:dimethyl ether (60:40) solvent.

The anti-inflammatory properties of oleocanthal have been registered in Patent Ep 2583676A1.

In another patent in America in 2011 (U.S. Pat. No. 7,879,344B2), the anti-inflammatory properties of oleocanthal (0.14%) in a topical drug carrier have been claimed. In 2020, in U.S. Pat. No. 6,437,004B1 US, the oleocanthal formulation has been claimed to protect the skin against oxidative stress in skin diseases including atopic dermatitis (eczema), contact dermatitis, rosacea, psoriasis, radiation burns, exiosis, etc.

### SUMMARY OF INVENTION

The invention entitled “Malt supplement Containing Oleocanthal, with Anti-inflammatory, Antioxidant and Anti-cancer Properties” is in the field of extracting and formulating a compound used in food, pharmaceutical, and supplement industries with special applications in biomedicine. Oleocanthal is a polyphenol with significant medicinal and nutritional value, however, no method has been proposed yet for its economic extraction while preserving properties. Moreover, this substance is lost in the debittering process and becomes useless. In this invention, an innovative supercritical process with triple solvents is used to extract quality oleocanthal from the olive fruits at a high rate and the temperature and pressure of 59° C. and 2 MPa, respectively. Interestingly, in this process, the materials used can be collected and used again. The addition of oleocanthal in the boiling stage of the malt extraction process leads to the production of a cost-effective and quality malt drink supplement with antioxidant and anti-cancer properties.

### Technical Problem

Oleocanthal is a valuable polyphenol whose extraction using the existing methods is not economically justifiable and in the process of debittering olives, it is removed along with other polyphenols. Loss of oleocanthal despite the increasing need for natural antioxidant and anti-cancer agents in diets, as well as failure to maintain its structure and properties in the extraction process, are the major problems. The main purpose of this invention is to provide an innovative method to extract, preserve, and increase the consumption of oleocanthal in malt supplement formulation.

### Solution to Problem

Until now, several methods have been proposed for the extraction of oleocanthal from virgin olive oil, none of which have been economical for the process of debittering olives.

In this method, olives are first pitted in the canning stage, and after washing, they are kept in distilled water for 24-48 hours. Then, the bitter water is collected and the rest of the ingredients enter the canning process. Bitter water is lyophilized after pasteurization, and the resulting powder contains polyphenols, chlorophyll, some water-soluble proteins, etc. This powder has been invented for long-term storage. To extract oleocanthal, the resulting powder is first dissolved in water again and after passing through activated carbon filters, chlorophyll and pigment are removed. After filtering, beta-glucan is added to the resulting solution and enters the two-step extraction as follows. In the first and second steps, propane:liquid carbon dioxide:hexane solvent is added to the above-mentioned solution at a ratio of (30:60:10) and (10:60:30), respectively with the supercritical method using the combination of propanol:ethanol (50:50) at 59° C. and 2 MPa. Then the supernatant is filtered and the solvent is removed by lyophilization, and oleocanthal is purified (FIG. 1).

After repeating step 2 for more purity, the extraction results are confirmed using HPLC. In order for the general public to benefit from the anti-cancer and anti-inflammatory properties of oleocanthal, this material will be used in the process of malt drink production. For this purpose, after fermentation, 1-5% oleocanthal is added at the boiling stage and after checking the Brix degree, the drink is bottled (FIG. 2).

To prevent the oxidation of oleocanthal, tocopherol (1:1000) is simultaneously added to malt.

### Advantageous Effects of Invention

Innovative extraction of oleocanthal polyphenol from olive fruits during the canning process

Making a natural supplement from a natural substance that is wasted.

Utilizing the antioxidant properties of oleocanthal in the food and supplement industries

Utilizing the anti-inflammatory and analgesic properties of oleocanthal

Utilizing the anti-cancer properties of oleocanthal

Utilizing the other properties including skin protection

Quick, easy, and cheap access to oleocanthal, a valuable substance in olive

### BRIEF DESCRIPTION OF DRAWINGS

FIG. 1: The process of extracting oleocanthal from olive fruit used for canning

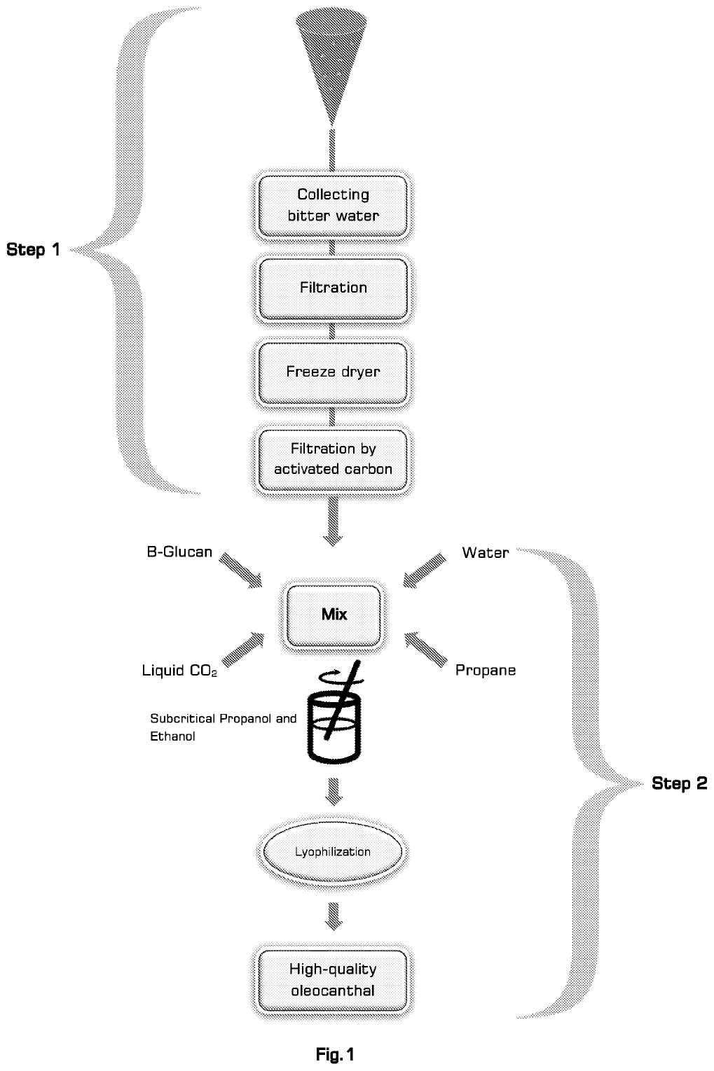

FIG. 2: The steps of producing malt supplement containing oleocanthal, with anti-inflammatory, antioxidant, and anti-cancer properties

### DESCRIPTION OF EMBODIMENTS

Extraction of oleocanthal from olive fruit in the canning stage. Olives are first pitted in the canning stage, and after washing, they are kept in distilled water for 24-48 h. Then, the bitter water is collected and lyophilized after pasteurization. The resulting powder containing polyphenols, chlorophyll, etc. is first dissolved in water again and after passing through the activated carbon filters, chlorophyll and pigment are removed. Then, beta-glucan is added to the resulting solution and enters the two-step extraction process. Propane:liquid carbon dioxide:hexane solvent is added to the solution at the ratios of (30:60:10) and (10:60:30), respectively with the supercritical method using the combination of propanol:ethanol (50:50) at 59° C. and 2 MPa. Then, the supernatant is filtered and the solvent is removed by lyophilization, and oleocanthal is purified.

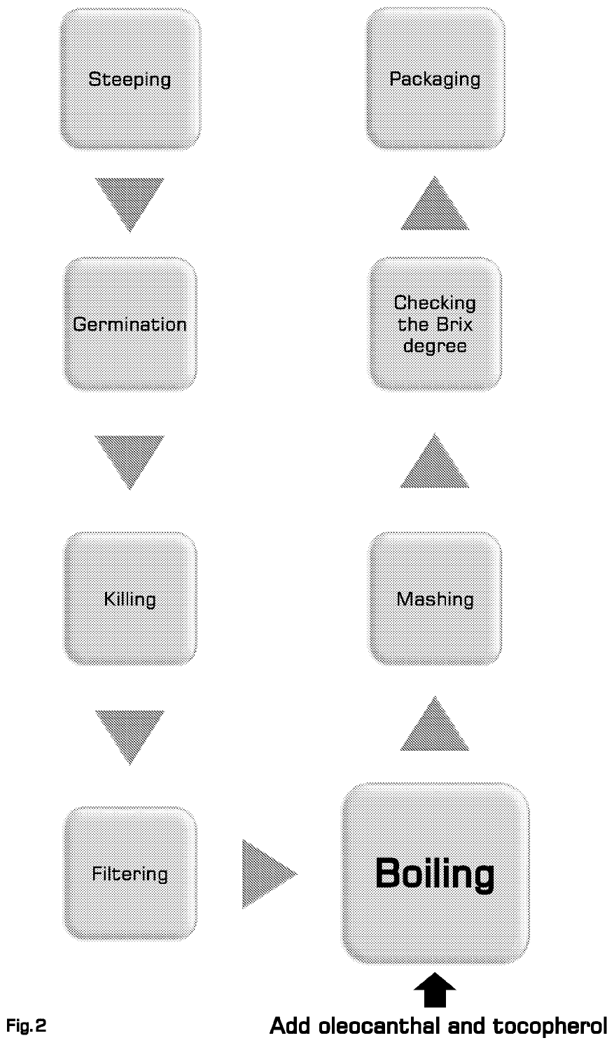

FIG. 2: The steps of producing malt supplements containing oleocanthal, with anti-inflammatory, antioxidant, and anti-cancer properties. The malting steps include sifting and cleaning, steeping, germination, killing, mashing, boiling, filtering, checking the Brix degree, and packaging, and oleocanthal is added in the final step (boiling) after fermentation.

### Industrial Applicability

Extraction of oleocanthal from olive fruits in the canning stage and producing oleocanthal supplement used in the food and pharmaceutical industries.

1. The extraction process includes binding oleocanthal to beta-glucan and two-step extraction, In the first and second steps, propane:liquid carbon dioxide:hexane solvent is added to the above-mentioned solution at a ratio of (30:60:10) and (10:60:30) with the supercritical method using the combination of propanol:ethanol (50:50) at 59° C. and 2 Mpa, Then the solvent is removed and oleocanthal is purified.

2. According to claim 1, the triple alcohol-gas-aqueous, as well as aqueous solvents, lead to the highest quality and efficiency.

3. According to claims 1 and 2, the invention components include malt, oleocanthal, and tocopherol, and also the process of adding the oleocanthal at a ratio of 1-10% in the boiling stage.

4. According to claim 2, the final product has higher stability and the properties listed in the “Advantages of the Invention” Section can be maintained for a long time.

5. According to claims 2 and 3, tocopherol as an antioxidant increases the stability of oleocanthal.

6. According to claim 2, any other drinks including wine and whiskey containing oleocanthal and tocopherol are claimed
